(19) United States
(12) Patent Application Publication
(10) Pub. No.: US 2015/0209186 A1
(43) Pub. Date: Jul. 30, 2015

(54) Therapeutic Wound Dressing with Solid Copper Material

(71) Applicants: Chun Lim Abbott, Pittsburgh, PA (US); Dominic C. Abbott, Pittsburgh, PA (US)

(72) Inventors: Chun Lim Abbott, Pittsburgh, PA (US); Dominic C. Abbott, Pittsburgh, PA (US)

(21) Appl. No.: 14/168,310

(22) Filed: Jan. 30, 2014

(51) Int. Cl. A61F 13/00; A61L 15/18; A61F 13/02

(52) U.S. Cl. CPC A61F 13/00063; A61F 13/0256; A61F 13/0206; A61L 15/18

(57) ABSTRACT

A therapeutic wound dressing comprises a sheet of tape having an adhesive surface, a pad secured on the adhesive surface and a strip of solid copper material secured on the tape so as to be disposed within the periphery of the pad. The adhesive surface allows the wound dressing to be releasably adhered to the skin to cover a wound on the skin with the copper material in contact with the wound to provide therapeutic effects.

### BACKGROUND OF THE INVENTION

1. Field of the Invention

The present invention pertains to wound dressings for protecting wounds on the skin. More particularly, the invention pertains to therapeutic wound dressings that have antimicrobial, anti-pathogenic and anti-inflammatory effects on wounds on the skin.

2. Brief Discussion of the Related Art

Microbes and pathogens such as bacteria, viruses and fungi commonly occur on skin surfaces of the body. Sometimes infections develop on the skin at the site of a cut, scratch, abrasion, burn, splinter, boil, pimple, blister, insect bite, animal bite, cysts, insect stings, sutures, ulcers or other wounds or trauma that damage or break the skin or otherwise provide a point of entry for bacteria and/or other harmful organisms. When a wound appears on the skin, it is common practice to apply a wound dressing on the skin to cover and protect the wound while the skin heals and/or to absorb secretions from the wound. Many types of conventional wound dressings are commercially available for this purpose. Other than providing a protective covering over the wound and/or absorbing secretions, conventional wound dressings are not of themselves therapeutic to the extent that they do not actively kill microorganisms and pathogens and do not actively reduce inflammation and, therefore, pain. For this reason, conventional wound dressings are typically used in conjunction with other products, i.e. topical substances that are applied to the wound or to the dressing in order to provide antibacterial and/or anti-inflammatory effects on the wound. Such products are oftentimes of limited effectiveness as they are in large part absorbed by the wound dressing itself and they dissipate quickly when applied to the wound.

It has previously been established that copper possesses properties by which it is capable of killing, neutralizing and preventing the growth of human pathogens. It is known that many bacteria identified as human pathogens cannot survive on surfaces of copper metal. U.S. Pat. No. 8,135,466 B2 to Fuller et al discloses a joint prosthesis having an implant body with an external surface containing an antimicrobial metal where the antimicrobial metal may be copper. U.S. Patent Application Publications No. US2012/0071807 A1 and No. US2012/0089068 A1 to McClure, Jr. disclose wound dressings containing a metal-based antimicrobial agent where the metal-based antimicrobial agent may be a mixture of silver ions and copper ions. Devices having an external surface of copper metal for insertion in the vagina to treat abnormal biological conditions have been proposed by Applicants in U.S. patent application Ser. No. 12/157,823 filed Jun. 13, 2008, Ser. No. 13/317,230 filed Oct. 12, 2011, and Ser. No. 13/464,005 filed May 4, 2012.

Topical substances containing particles of copper or its alloys have been proposed for health support uses. A product called “MesoCopper®” sold by Purist Colloids, Inc. is a colloidal copper solution containing nano particles of copper for use on the skin to minimize the appearance of fine lines and wrinkles. Another version of the product is sold as an ingestible mineral supplement. Copper peptides for use on the skin are also commercially available, i.e. small fragments of protein that have an affinity for copper to which they bind very tightly. U.S. Pat. No. 7,776,915 B2 to Morariu discloses a topical composition containing, at a minimum, a lipoic acid, a carnitine and a carnosine, where the carnosine may be chelated to zinc or copper ions. The intended use for the topical composition is to improve the appearance of aged skin. U.S. Patent Application Publication No. US 2008/0195033 A1 to Eagleson et al discloses use of a metal substance to treat diseases in the body. The metal substance is primarily a colloidal suspension, and delivery of the substance to the body may require the use of electricity.

Prior to the present invention, it has not been recognized to provide a wound dressing having a strip of solid copper material for being placed in contact with a wound on the skin, whereby the copper material in contact with the wound has antimicrobial, anti-pathogenic and anti-inflammatory therapeutic effects on the wound.

### SUMMARY OF THE INVENTION

A therapeutic wound dressing comprises a sheet of tape having an adhesive surface, a pad secured on the adhesive surface and a strip of solid copper material secured on the tape so as to be disposed within the periphery of the pad. The adhesive surface allows the wound dressing to be releasably adhered to the skin to cover a wound on the skin such that the strip of solid copper material is in contact with the wound. The pad preferably has a periphery and size to fit entirely within the confines of the periphery of the tape such that a peripheral border of the adhesive surface circumscribes the periphery of the pad. In this way, the entire periphery of the wound dressing can be adhesively sealed to the skin to deter the entry of foreign substances. The pad may be made of any suitable material, preferably a soft, compressible and absorbent material. The strip of solid copper material is made from solid copper metal, preferably pure copper. The strip of solid copper material preferably has a peripheral configuration and size to fit entirely within the confines of the periphery of the pad such that a peripheral border of the pad circumscribes the periphery of the strip of solid copper material. The copper material in contact with the wound has antimicrobial, anti-pathogenic and anti-inflammatory effects on the wound so as to kill or neutralize potentially harmful pathogens, reduce the risk and occurrence of infection, promote healing, and reduce pain.

Other aspects and advantages of the present invention will become apparent from the following description of the preferred embodiments taken in conjunction with the accompanying drawings wherein like parts in each of the several figures are identified by the same reference characters.

### BRIEF DESCRIPTION OF THE DRAWINGS

FIG. 1 is a plan view of a therapeutic wound dressing with solid copper material.

FIG. 2 is an exploded view of the therapeutic wound dressing of FIG. 1.

FIG. 3 is a sectional view of the therapeutic wound dressing.

FIG. 4 is an exploded view of an alternative therapeutic wound dressing with solid copper material.

FIG. 5 is a sectional view of the alternative therapeutic wound dressing of FIG. 4.

FIG. 6 is a perspective view depicting the therapeutic wound dressing being applied to a wound on the wrist.

FIG. 7 is a perspective view depicting the therapeutic wound dressing being applied to a wound on the finger and illustrating an alternative configuration for the therapeutic wound dressing.

### DETAILED DESCRIPTION OF THE INVENTION

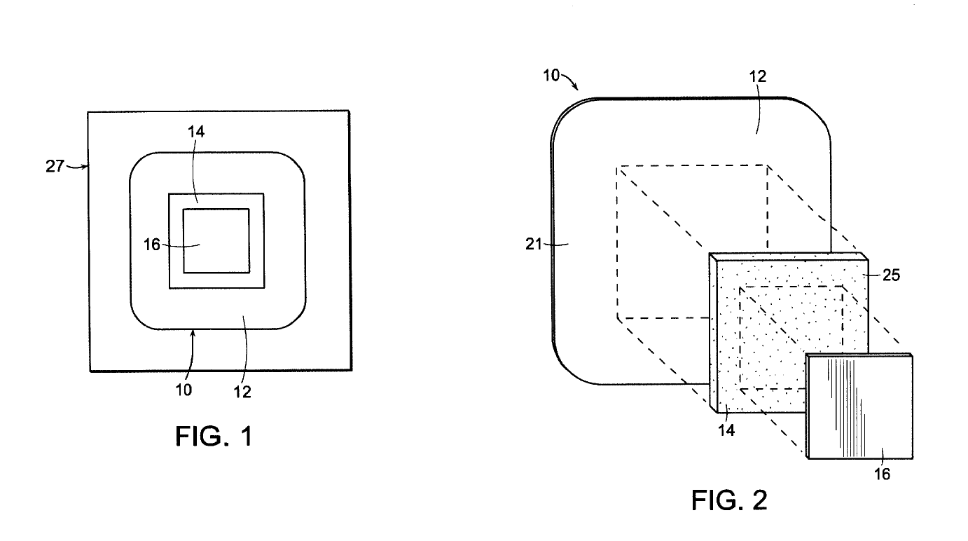

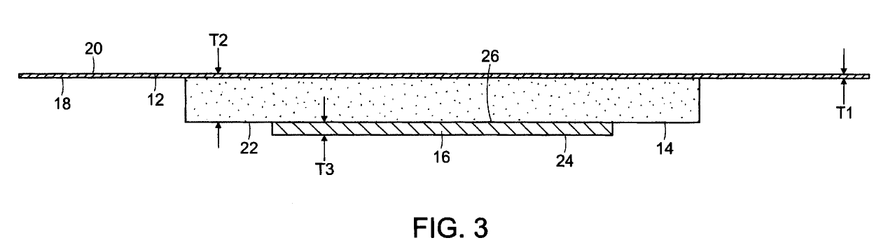

FIGS. 1-3 depict a therapeutic wound dressing with solid copper material 10 comprising a sheet of adhesive tape 12 for use on the skin, a pad 14 secured to the tape 12, and a sheet or strip of solid copper material 16 mounted on the tape 12. The sheet of tape 12 is of any suitable peripheral configuration and area to cover a wound on the body and is preferably of minimal thickness T1, for example, ¼ mm thick. In the case of wound dressing 10, the sheet of tape 12 is of generally square peripheral configuration having a length of about 2 inches and a width of about 2 inches. As shown in FIG. 2, the peripheral configuration of tape 12 may have rounded or radiused corners. As shown in FIG. 3, one surface or side 18 of the sheet of tape 12 is an adhesive surface for being applied to and releasably adhered to skin while the opposite surface or side 20 of the tape 12 is a non-adhesive surface that is preferably water-resistant. The adhesive surface 18 thus constitutes a skin-contacting or skin-facing side of the dressing 10. The tape 12 may be any suitable medical grade adhesive tape or adhesive material used for releasable adhesion on the skin to temporarily cover and protect a wound.

One surface or side of the pad 14 is secured on the adhesive surface 18 of the tape 12. The pad 14 has a peripheral configuration and area to fit entirely within the confines of the periphery of tape 12 in spaced relation, such that a peripheral border 21 of the adhesive surface 18 of tape 12 circumscribes or surrounds the entire periphery of the pad 14. As shown in FIGS. 1 and 2, the pad 14 is of square peripheral configuration having a length of about 1% inches and a width of about 1½ inches. The pad 14 is centered with respect to the periphery of the tape 12 in spaced relation such that a peripheral border 21 of ¼ inch between the periphery of the pad and the periphery of the tape of the adhesive surface 18 circumscribes the periphery of the pad 14. It should be appreciated that the pad 14 can have a peripheral geometric configuration that is different from the peripheral geometric configuration of the tape 12 and that the size of the peripheral border 21 can vary. The pad 14 can be made of any suitable material, such as sterile gauze, typically used as a dressing for a wound. The pad 14 is preferably soft, compressible and absorbent. The pad 14 can be of suitable thickness T2, such as ⅛ inch in thickness. The adhesive on the adhesive surface 18 of the tape 12 can be used to secure the pad 14 thereon without the necessity for additional adhesive. It should be appreciated, however, that additional adhesive between the pad 14 and the tape 12 could be provided if necessary. The surface or side 22 of pad 14 that is opposite the side that is secured to the adhesive surface 18 of the tape 12 is thus exposed along the skin-contacting side of the dressing 10 and it constitutes a skin-contacting or skin-facing side of the pad 14.

The strip of solid copper material 16 is secured on the pad 14 along the skin-contacting side of the dressing 10. More specifically, one surface or side of the strip of solid copper material 16 is secured to the skin-contacting side 22 of pad 14. The opposite surface or side 24 of the strip of solid copper material 16 is thus exposed along the skin-contacting side of the wound dressing and constitutes a skin-contacting or skin-facing side of the strip of solid copper material 16. The strip of solid copper material 16 has a peripheral configuration and area to fit entirely within the confines of the periphery of pad 14 in spaced relation, such that a peripheral border 25 of the pad 14 circumscribes or surrounds the entire periphery of the strip of solid copper material 16. In the embodiment illustrated in FIGS. 1-3, the strip of solid copper material 16 has a square peripheral configuration with a length of 1 inch and a width of 1 inch. The strip of solid copper material 16 is centered with respect to the periphery of the pad 14 in spaced relation such that a peripheral border 25 of ¼ inch of the pad 14 circumscribes the periphery of the strip 16. It should be appreciated, however, that the strip of solid copper material 16 can have a peripheral geometric configuration different from that of the tape 12 and/or the pad 14 and that the size of the peripheral border 25 can vary. The strip of solid copper material 16 is preferably of minimal thickness T3, such as 1 mm in thickness. The solid copper material is a solid copper metal and, preferably, is pure copper (99.5 percent or greater copper). As seen in FIG. 3, the strip of solid copper material 16 is secured to the skin-facing side 22 of the pad 14 using a thin deposition or layer of adhesive 26 between the strip of solid copper material 16 and the skin-facing side 22 of the pad 14. The skin-contacting side 24 of the strip of solid copper material 16 is thus exposed along the skin-contacting side of the dressing 10 and its periphery is circumscribed by the peripheral border 25 of the pad 14 which, in turn, is circumscribed by the peripheral border 21 of the adhesive surface 18 of tape 12.

The wound dressing 10 will normally be supplied for use in a sterile, sealed package 27 as seen in FIG. 1. The package 27 can be made of any suitable material and can have front and back walls sealed along their peripheral edges with the dressing disposed therebetween.

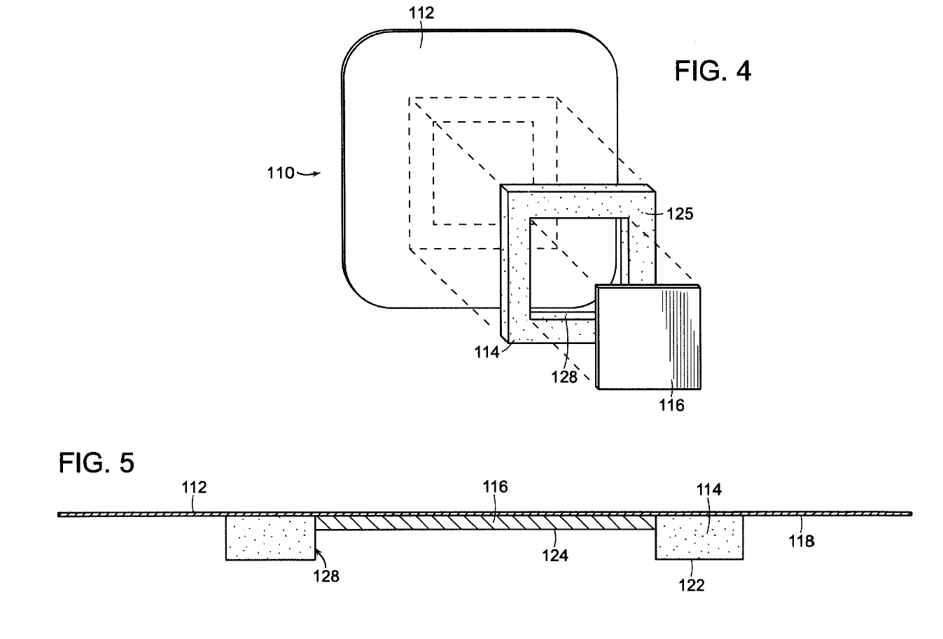

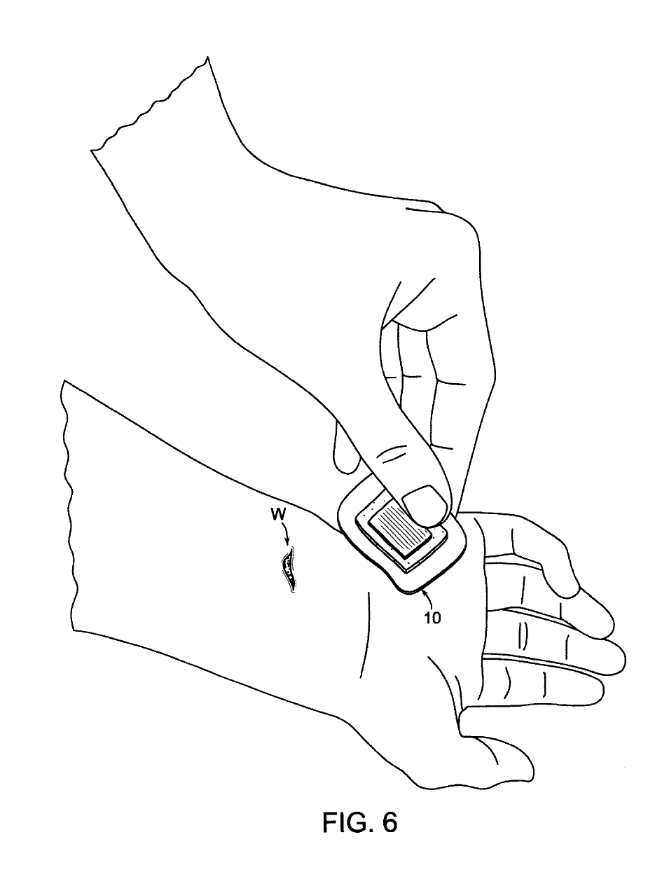

FIGS. 4 and 5 illustrate an alternative wound dressing 110 comprising the sheet of tape 112, the pad 114 and the strip of solid copper material 116. The wound dressing 110 is similar to wound dressing 10 except that pad 114 has a window or aperture 128 therein circumscribed by the peripheral border 125. The size and configuration of the aperture 128 corresponds to the peripheral size and configuration of the strip of solid copper material 116 so that the strip 116 fits within the aperture 128 in a mating relation or fit. In this embodiment, one side of the strip of solid copper material 116 is secured to the adhesive surface 118 of the tape 112 without the pad 114 interposed therebetween. The adhesive on the tape 12 may provide sufficient holding force to secure the strip of solid copper material 116 thereto without the need for additional adhesive. However, it should be appreciated that an additional adhesive may be interposed between the strip of solid copper material 116 and the adhesive surface 18 of the tape 12 if necessary. As seen in FIG. 5, this embodiment allows the skin-contacting side 124 of the strip of solid copper material 116 to be flush with or slightly recessed relative to the skin-contacting side 122 of the pad 114. Since the pad 114 may be made of compressible material, such as gauze, good contact between the skin-contacting surface 124 of the copper and the skin is ensured when the wound dressing is applied as described further below. Normally, the wound dressing 110 will be provided for use in a sterile package as explained above.

In use, the package 27 is opened and the wound dressing 10 is removed therefrom. The thusly exposed skin-contacting side of the wound dressing is then placed in contact with a wound located on the skin of the body. The wound dressing 10 is positioned on the skin of the body such that the skin-contacting side or surface of the strip of solid copper material 16 is in direct contact with the wound. Depending on the size of the wound, a wound dressing 10 would be selected that has a strip of solid copper material 16 of appropriate size to cover the entire wound or as much of the wound as possible. The peripheral border 25 of the pad 14 that circumscribes the strip of solid copper material 16 assists in preventing any discomfort that the peripheral edge of the strip 16 may cause when in contact with the skin. In addition, the peripheral border 25 of the pad 14 assists in protecting the wound and/or the area of skin surrounding the wound, and assists in absorbing any secretions that may emanate from the wound. The peripheral border 21 of the adhesive surface 18 of tape 12 serves to secure the wound dressing 10 to the skin surrounding the wound. In use, the periphery of the wound dressing 10 would be gently pressed against the skin to assist in ensuring that the adhesive border 21 is firmly secured to the skin. The adhesive border 21 of tape 12 that is adhesively secured to the skin also serves to deter the entry of foreign substances between the skin-contacting side of the wound dressing 10 and the wound. FIG. 6 depicts the wound dressing 10 in the process of being applied to the skin so that the skin-contacting side 24 of the strip of solid copper material 16 is in direct contact with a wound W on the wrist of a person.

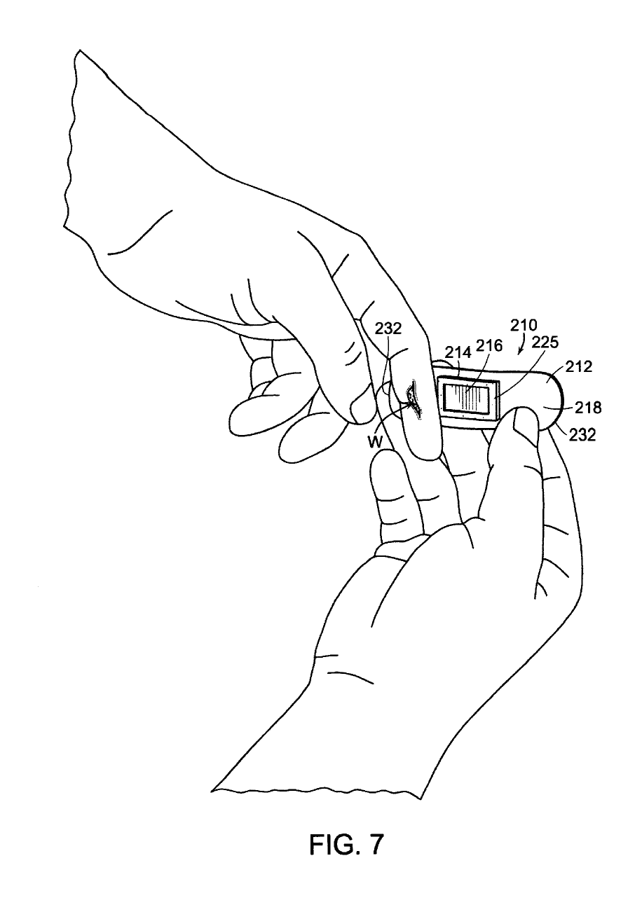

FIG. 7 depicts an alternative wound dressing 210 being applied to the finger of a person to cover a wound W on the finger. The wound dressing 210 comprises an elongate strip of tape 212 having the adhesive surface 218, a pad 214 centered on the adhesive surface 218, and the strip of solid copper material 216 centered on the pad 214. In this embodiment, the peripheral border 225 of pad 214 circumscribes the periphery of the strip 216 and the adhesive surface 218 of the tape 212 circumscribes the periphery of the pad 214. However, the elongate configuration of the tape 212 provides end tabs 232 of tape 212 that extend respectively from opposite sides of the pad 214 and along which the adhesive surface 218 is exposed. The wound dressing 210 is particularly well suited for being wrapped around a finger or similar anatomical structure, the adhesive surface 218 along the tabs 232 being used to secure the wound dressing 210 to the anatomical structure with the skin-contacting side of the strip of solid copper material 216 in direct contact with a wound W along the skin on the anatomical structure. The wound dressing 210 would normally be provided for use in an appropriate package.

The therapeutic wound dressing can be used on many various types of wounds, arising from internal or external causes, including cuts, scratches, abrasions, burns, splinters, boils, pimples, blisters, insect bites, animal bites, cysts, insect stings, sutures, ulcers and any other wounds or trauma that damage or break the skin or otherwise provide a point of entry for bacteria and/or other harmful organisms. The strip of copper material in contact with the wound has an antimicrobial, anti-pathogenic and anti-inflammatory effect on the wound. In particular, the copper material serves to kill or neutralize potentially harmful organisms such as bacteria, viruses and fungi, such that the risk and occurrence of infections at the site of the wound is reduced. In addition, the anti-inflammatory effect on the wound provided by the copper material accelerates healing and provides pain relief.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. A therapeutic wound dressing for being applied to the skin to cover a wound on the skin comprising
a sheet of tape having an adhesive surface for being releasably adhered to the skin, said sheet of tape having a periphery;
a pad secured on the adhesive surface of the tape and having a periphery disposed entirely within said periphery of said tape; and
a strip of solid copper material secured to the tape and having a periphery disposed entirely within said periphery of said pad, whereby said copper material in contact with the wound has antimicrobial, anti-pathogenic and anti-inflammatory effects on the wound.

2. The therapeutic wound dressing recited in claim 1 wherein said pad is secured to said adhesive surface of said tape such that a peripheral border of said adhesive surface of said tape circumscribes said periphery of said pad.

3. The therapeutic wound dressing recited in claim 2 wherein said strip of solid copper material is secured to said tape such that a peripheral border of said pad circumscribes said periphery of said strip of solid copper material.

4. The therapeutic wound dressing recited in claim 3 wherein said strip of solid copper material is secured to said pad with an adhesive disposed between said pad and said strip of solid copper material.

5. The therapeutic wound dressing recited in claim 3 wherein said pad has an aperture therein circumscribed by said peripheral border of said pad and said strip of solid copper material fits within said aperture and is secured to said adhesive surface of said tape.

6. The therapeutic wound dressing recited in claim 1 wherein said strip of solid copper material has a periphery and area to cover the wound in its entirety.

7. The therapeutic wound dressing recited in claim 1 wherein said copper material is pure copper metal.

8. The therapeutic wound dressing recited in claim 1 wherein said pad is made of a soft, compressible material.

9. The therapeutic wound dressing recited in claim 1 and further comprising a sterile package which said therapeutic wound dressing is disposed prior to use.
